(19) United States
(12) Patent Application Publication
(10) Pub. No.: US 2005/0204612 A1
(43) Pub. Date: Sep. 22, 2005

(54) Method and device for producing biodiesel

(76) Inventors: Joosten Connemann, Leer (DE); Jurgen Fischer, Hamburg (DE); Hans Groos, Aurich (DE); Arne Philippsen, Leer (DE)

Correspondence Address: OSTROLENK FABER GERB & SOFFEN, 1180 AVENUE OF THE AMERICAS, NEW YORK, NY, 100368403, US

(21) Appl. No.: 10/528,333

(22) PCT Filed: Sep. 8, 2003
(86) PCT No.: PCT/EP03/09965

§ 371 Date: Jun. 2, 2005

(30) Foreign Application Priority Data
Sep. 20, 2002 (DE) 102 43 700.9

(51) Int. Cl. C10L 1/18; C07C 51/43

(52) U.S. Cl. 44/437; 554/174

(57) ABSTRACT

The present invention concerns a method for the continuous production of biodiesel from biogenic fat- or oil-containing starting mixtures with a high content of free fatty acids as well as a device for producing biodiesel.

The present invention concerns a process for the continuous production of biodiesel from biogenic initial feedstock mixtures containing fat or oil with a high content of free fatty acids, as well as a device for the production of biodiesel.

For a long time already, there has been a growing interest in substituting petroleum-based fuels with inexpensive and environmentally friendly alternatives. The ever-growing number of passenger cars and lorries, coupled with diminishing petroleum resources and the higher production costs resulting therefrom, served as the impetus in seeking alternative fuels. Plant-based oils are one of the possible alternatives, whereby in Central Europe, rapeseed oil is mainly being considered as the initial product.

Prior to being used as fuel for diesel engines, plant-based oils must be cleaned via extensive process steps. Lecithins, carbohydrates and proteins are removed from the oils via the introduction of phosphoric acid or citric acid. Subsequently the oil thus treated is centrifuged so as to remove the so-called oil gums. An extraction via caustic soda is carried out in order to remove the free fatty acids from crude rapeseed oil.

Plant-based oils differ significantly in several points from the technical properties of diesel fuels. They possess, for example, a greater density than diesel fuel. The cetane number (ability to ignite) of rapeseed oil is lower than that of diesel fuel. Thus, the engine may run rather unevenly, with a noteable increase in noise emission. Furthermore, the viscosity of rapeseed oil is much higher than that of diesel fuel. The result of the higher viscosity of rapeseed oil is a more inefficient spray and combustion within the combustion chamber. For conventional engines, the use of purely plant-based oils results therefore in the build-up of coke residue, together with an increased particle emission.

Some of the problems that occur when vegetable oils are used in engines that have not been adapted accordingly, can be solved by converting the triglycerides or fatty acid esters of glycerine into fatty acid alkyl esters, especially methyl or ethyl esters. Thus, these esters, also termed as biodiesel, can be used in a relatively problem-free manner for non-modified diesel engines, whereby the emission of unburnt hydrocarbons and soot particles can be considerably reduced compared to conventional diesel fuel.

However, biodiesel that is produced from vegetable oils is relatively expensive due to the price of raw materials and the required refinery processes, thus the price of biodiesel is not able to compete with the price of conventional diesel fuel. Therefore, the utilization scope of biodiesel as a fuel for conventional diesel engines is considerably reduced. In order to establish biodiesel as a fuel that is able to compete in the long-term with conventional diesel fuel, it is of the utmost importance to lower its production costs considerably. One of the means of reducing production costs is the use of initial materials that are much cheaper than rapeseed oil.

Some of these alternative raw materials are, for instance, used oils from restaurants and oils and fats of animal origin. Approximately 1.1 million tons per year of the above raw materials are estimated to be available in the US, for example (Haumann, Inform, 1 (8) (1990), 722-725). However, the problem inherent in the processing of such used oils and fats is that they contain large amounts of free fatty acids that form soaps during esterification when base catalysts are being used. These resulting soaps may interfere to a considerable degree with the separation of the biodiesel from the glycerine formed as a by-product during base transesterification. Therefore, the use of acid catalysts that do not result in any soap formation was suggested (Aksoy et al., JAOCS, 65 (1988), 936-938).

Studies by Canakci and Van Gerpen (Transactions of ASAE, 42 (5) (1999), 1203-1210) have shown that acid catalysts can convert free fatty acids into their esters, but are hardly suitable for the conversion of triglycerides into biodiesel.

Several processes were developed for the production of biodiesel from used oil and fat containing initial materials. Haas et al. describe a process for the production of biodiesel from soapstock, whereby initially all fatty acyl ester compounds of the initial feedstock are hydrolyzed via saponification. Following the removal of water, the resulting free fatty acids are subjected to acid esterification (Haas et al., Energy & Fuels, 15 (2001), 1207-1212). A disadvantage of this process lies in the fact that in relation to the fatty acid mixture, relatively large amounts of methanol and sulphuric acid have to be used (molar ratio of fatty acid mixture to methanol to sulphuric acid approximately 1:30:5, or in relation to weight approximately 1:4:2), which can no longer be regarded as economical.

In an alternative process, the free fatty acids of the initial feed stock are first converted into esters via acid esterification, and the fatty acid triglycerides are subsequently subjected to a base transesterification (Canakci and Van Gerpen, 1999). The process is designed in such a way that for a feed stock with the acid number of 66, 10% sulphuric acid and approximately 400% methanol in relation to weight are used (molar ratio of methanol to fatty acid mixture 40:1), in order to thereby obtain esterification to an acid number of 2.0. In this process, the yields vary between 75% and 80%. It is a considerable disadvantage of this process that the water formed during esterification of the free fatty acids with alcohol may greatly impair or even completely prevent the subsequent base transesterification.

The overall results show that biodiesel produced from used fats and oils frequently does not conform to the demands required of fuels. For example, the content of free and bound glycerine is much too high in some of the biodiesels produced according to the state-of-the-art-technology based on used oils and fats. It was also found that the composition of the produced biodiesel is subject to strong fluctuations dependent on the initial feedstock.

It is therefore the task of the present invention to propose a solution to the technical problem of presenting non-costly processes and means for the production of biodiesel based on different biogenic starting materials containing oils and fats, especially used oils and fats, whereby a more efficient conversion of the initial feedstock into biodiesel is possible, and whereby a biodiesel fuel conforming to standards is produced that can be used directly as fuel, without having to undergo further processing steps.

The present invention solves the underlying problem by means of a non-pressurized process for the continuous production of alkyl esters of higher fatty acids, especially biodiesel, from fatty acid triglyceride starting feedstock containing free fatty acids, with the integrated combination of acid esterification and base transesterification, comprising
a) single or multiple esterification of the free fatty acids in separate esterification devices connected with each other, with a C₁- to C₄-mono-alcohol in the presence of an acid catalyst and with glycerine as a
dragging agent, at 60° C. to 65° C. while an esterification mixture is produced,
b) partial purification of the esterification mixture via partial separation of the dragging agent, acid catalyst and unconverted C₁- to C₄-mono alcohol,
c) transesterification of the fatty acid triglycerides, carried out at least twice, in separate transesterification devices connected with each other, with a C₁- to C₄-mono alcohol in the presence of a base catalyst at 60° C. to 65° C. while a transesterification mixture is produced, and
d) purification of the transesterification mixture via separation of the base catalyst, unconverted C₁- to C₄-mono alcohol and the glycerine produced during transesterification by means of a treatment using water inside at least one separator with subsequent drying,
characterised by the fact that the C₁- to C₄-mono alcohol used for esterification, the dragging agent glycerine used for esterification and the water used for purification of the transesterification mixture are at least partially recovered from the esterification and transesterification mixtures and that after purification, the acid and base catalysts from the esterification and transesterification mixtures are converted, resulting in the formation of a salt suitable for use as a fertilizer.

The present invention further solves the underlying technical problem by presenting a device for carrying out the process according to the invention, i.e. a device provided with an integrated combination of units for acid esterification and for base transesterification.

Due to the process according to the invention and the device according to the invention for carrying out the above process, it is possible, in a simple and inexpensive manner, to produce biodiesel from a number of purified or unpurified starting materials containing oil and/or fat. Preferably, used oils or fats in particular with different contents of free fatty acids, such as oils that were used for deep-frying food, industrial tallow, abattoir waste, etc. are used, which are extremely cheap when compared with the starting materials such as rapeseed oil, that are commonly used for the production of biodiesel.

These starting materials, after undergoing a preliminary purification process, if necessary, in order to remove mucous matter or non-soluble particles such as bones, etc., are subjected at least once and preferably several times to an acid esterification process with a C₁- to C₄-mono alcohol while using an acid catalyst and a dragging agent. The free fatty acids contained in the starting materials are almost completely converted into their respective alkyl esters due to being subjected to acid esterification once or several times.

After removal of at least part of the non-converted mono alcohol, the acid catalyst used and the dragging agent, the esterification mixture obtained in this way, which contains the alkyl esters of the free fatty acids and the oils and fats now present as fatty acid triglycerides, is then directly subjected, at least twice, to a base transesterification process with the same C₁- to C₄-mono alcohol in the presence of a base catalyst while releasing glycerine. Due to the repeated base transesterification, almost all fatty acid triglycerides contained in the starting mixture are converted into the alkyl esters of the higher fatty acids bound in the triglycerides. Thus a transesterification mixture is obtained that comprises the alkyl esters of the free fatty acids and the alkyl esters that were bound as glycerides in the initial material. An almost 100% conversion into biodiesel of the initial mixtures used is obtained via the repeated esterification and transesterification reactions that were carried out in succession.

Due to the process according to the invention and the device according to the invention for carrying out the above process, it is possible to use several different starting materials, each with a different content of free fatty acids, simultaneously and in an advantageous manner. According to the invention, it is possible to first subject a starting material with a high content of free fatty acids to an esterification process. The esterification mixture thus obtained may then be mixed in an advantageous manner with a starting mixture inherently containing hardly any fatty acids, and may then be transesterified. Naturally it is also possible to mix such different starting materials before carrying out the process according to the invention and then to esterify these mixtures together prior to transesterification of the final mixture.

The number of esterification and transesterification reactions to be carried out depend especially on the characteristics of the starting mixtures, such as their composition and content of free fatty acids and fatty acid triglycerides, as well as on the purity of the starting mixtures. According to the invention it is intended, for example, that an starting material with a high content of free fatty acids and a low content of fatty acid triglycerides is subjected to more esterification reactions and fewer transesterification reactions than a starting material with a lower content of free fatty acids and a higher content of fatty acid triglycerides. An important advantage of the device according to the invention is the fact that the device according to the invention can be modified in a very simple manner depending on the composition of the different starting materials that are to be converted. A preferred embodiment of the invention provides, for example, that the device according to the invention includes a total of approximately 10 columns for esterification and transesterification of the starting materials. If the starting material has a very high content of free fatty acids but a very low content of fatty acid triglycerides, it is possible that 8 of the columns are used for acid esterification, for example, while only 2 columns are used for base transesterification. However, if the initial material has a relatively low content of free fatty acids and a relatively high content of fatty acid glycerides, four of the columns, for example, may be used for esterification while four to six columns may be used for base transesterification. According to the invention it is also possible to completely deactivate some of the columns of the device according to the invention and to carry out fewer esterification and/or transesterification reactions. The device according to the invention thus permits the desired arrangement of the columns according to requirement, depending in particular on the fatty acid content of the starting materials used.

The biodiesel produced according to the invention possesses a high degree of purity with the lowest possible acid number and an extremely low glycerine content, and fulfills all quality criteria demanded of a biodiesel. Thus the quality criteria demanded according to the European Fuel Standard EN 14214, for example, are met, with the exception of the so-called CFPP (cold flow properties), which is a factor inherent in the raw material used and can only be influenced via suitable additives.

Due to the process according to the invention for the production of biodiesel and the device according to the invention for carrying out the above process, it is possible to effect, in an advantageous manner, considerable energy savings and reductions of the materials used, compared to the processes or devices, respectively, for the production of biodiesel that are already known in the state-of-the-art. The energy savings are due to the relatively low reaction temperatures of 60° C. to 65° C., as well as in particular from the fact that the individual reaction steps are carried out at ambient pressure, i.e. there is no energy required for the generation of a higher pressure.

Material savings are, for example, due to the fact that a constantly-to-be-renewed partial flow of the catalyst cycle of the esterification process is simultaneously used for neutralising the base catalyst of the transesterification process, and also due to the fact that the methanol ratio required, in relation to the initial amount of fatty acid mixture, is only approximately 25%, whereby the yield of the entire process is still 100%.

Savings of the materials used, i.e. the materials and agents used for carrying out the individual reaction steps, are also due in particular to the fact that the process according to the invention and the device according to the invention permit, in an especially advantageous manner, the purification and recovery of the used materials and agents, with the result that they can be recycled in the appropriate steps of the reaction process. It is an advantage that the purification steps of the process according to the invention are carried out within the cycle, so that multiple recycling of the purified agents is possible. For example, glycerine, the dragging agent used during acid esterification, can be almost completely recovered from the mixtures formed during esterification and transesterification. The C₁- to C₄-mono alcohol not converted in the course of the reactions is also recovered from the esterification and transesterification mixtures and is recycled, particularly during acid esterification. Water used during the purification steps or reaction water obtained during acid esterification can be reintroduced into the cycle after purification for recycling, thus reducing water consumption. Furthermore, due to the process according to the invention, the safe disposal of the acid and base catalysts utilized for esterification or transesterification reactions is possible in an advantageous manner, whereby the above catalysts are obtained from the conversion mixtures and are converted together, resulting in the formation of salts that can be used as fertilizer. This means that the process according to the invention for producing biodiesel and the device according to the invention for carrying out the above process serve to produce fertilizer at the same time.

The process according to the invention for producing biodiesel and the device according to the invention thus provide for an especially environmentally friendly production of biodiesel. On one hand, materials of ecological concern can be recycled, while on the other hand the agents and materials utilized during production are recovered and also recycled, so that the environment is not further burdened with harmful substances.

Thus, the process according to the invention for producing biodiesel aims to convert fat and/or oil containing initial mixtures with free fatty acids into biodiesel via multiple acid esterification and multiple base transesterification.

In conjunction with the present invention, “biodiesel” is the term for a mixture of mono alkyl esters of fatty acids that is obtained from oil and/or fat containing starting mixtures and can be used directly as a fuel for diesel engines.

“Fatty acid triglyceride starting mixtures containing free fatty acids” is the term for compositions that contain fats and oils as well as free fatty acids and that can be converted into biodiesel via esterification and/or transesterification.

According to the invention it is intended that the portion of fats and oils of the total composition is 0% to 100%, preferably at least 20%, more preferably at least 30% and most preferably more than 40%, and that the portion of free fatty acids is 100% to 0%, preferably at least 1%. “Free fatty acids” are unsaturated or saturated alkane carbonic acids with a non-branched chain of carbon that are not chemically bound in the starting mixtures. Fatty acids with 1 to 7 C-atoms are known as lower fatty acids, fatty acids with 8 to 12 C-atoms are known as medium fatty acids, while fatty acids with more than 12 C-atoms are known as higher fatty acids. According to the invention, the free fatty acids concerned may either be lower, medium-high or higher fatty acids.

“Fats and oils” is the term for solid, semi-solid or liquid fatty acid triglycerides, obtained from plants or animals, that essentially consist, in a chemical context, of mixed glycerine esters of higher fatty acids with an even number of carbon atoms. Fats and oils are water-insoluble and always have a lower density than water. While vegetable oils contain almost exclusively even-chained fatty acids, animal fats and oils also contain fatty acids with an uneven carbon number. The unsaturated fatty acids found in vegetable oils are present in a cis-form, while animal fatty acids are frequently present in a trans-configuration.

According to the invention it is intended that especially used or unused, unpurified or purified plant or animal based or industrial oils or fats or mixtures thereof, with a free fatty acid content of 0% to 100%, preferably of more than 1%, can be used as starting mixtures. “Used fats and oils” is the term for fat or oil containing materials that were used, after being obtained from appropriate vegetable or animal starting materials, especially for industrial purposes or for food production purposes, and which may be chemically modified or not modified due to their prior use, or may contain further additives that are particularly relevant to their prior use. “Virgin fats and oils” is the term for fat or oil containing materials which have not been used for any industrial purpose or for the purpose of food production after being obtained from the appropriate vegetable or animal starting materials, and which therefore contain only those substances that are inherent in the starting materials or are present due to the extraction process of the fats and oils from the starting materials. “Purified” means that prior to their utilization in the process according to the invention, the vegetable and animal or industrial oils and fats were subjected to preliminary treatment for the purpose of being purified and/or for concentrating the free fatty acids or fatty acid triglycerides, in order to remove other substances, such as proteins, colorants, etc. Thus “unpurified” means that the vegetable or animal or industrial oils and fats were not subjected to such preliminary treatment prior to their use in the process according to the invention. “Vegetable oils” are such oils and fats that are mainly obtained from vegetable starting materials, such as seeds, roots, leaves or other suitable parts of plants. Animal fats or oils originate mainly from animal starting materials, such as animal organs, tissue or other animal parts or animal body fluids such as milk. “Industrial oils and fats” are those oils and fats that were obtained in particular from vegetable or animal starting materials and were further processed for industrial purposes.

The used or unused, unpurified or purified oils and/or fats utilized according to the invention have been selected in particular from the group comprising soapstock, brown grease, yellow grease, industrial tallow, industrial lard, oil used for deep-frying, animal fat, edible tallow, vegetable crude oils, such as crude palm oil, animal crude oils or fats or mixtures thereof.

“Soapstock” is the term for a by-product obtained during the processing of vegetable oils, in particular a by-product of edible oil refineries based on soy, rapeseed or sunflower oil. The free fatty acid content of soapstock is approximately 50% to 80%.

“Brown grease” is the term for a waste product containing animal fat, whereby the free fatty acid content of brown grease is more than 15% to 40%. “Yellow grease” contains approximately 5% to 15% free fatty acids.

“Industrial tallow” and “industrial lard” are animal fats that are produced for industrial purposes and are obtained after a dry or wet melting process from abattoir waste, for example. Industrial tallow is evaluated and marketed according to its acid number, whereby the free fatty acid content varies between 1 and 15 to 20 weight %, and sometimes even higher, depending on the quality of the tallow. In general, industrial tallow has an acid number of approximately 10 to 40, or even higher. Industrial tallow includes, among others, industrial beef tallow, US fancy tallow, US bleachable fancy tallow, US special tallow, US tallow A and animal carcass fats.

“Animal fats” include fat-containing by-products that are obtained in particular as waste products during the processing of fowl, beef, pork, fish and sea mammal carcasses, such as solar stearin which is a solid residue remaining after lard oil has been obtained from pork lard via applied pressure. “Unpurified crude vegetable oils” are liquid or solid compounds, obtained via applied pressure from vegetable starting materials, that have not been subjected to any further processing other than being left to settle for the periods generally used for such purposes and being subjected to centrifugal separation or filtration, whereby only mechanical forces such as gravity, centrifugal forces or pressure are used to separate the oil from solid matter.

“Unpurified crude vegetable oils” may also include liquid or solid vegetable oils obtained via extraction, as long as these do not differ with regard to smell, colour or taste nor by any special established analytical data from the corresponding vegetable oils that were obtained via applied pressure. The free fatty acid content of unpurified vegetable oils and fats is variable. Thus, crude palm oil has a free fatty acid content of approximately 5% to approximately 15%. The “unpurified crude vegetable oils” also include, for example, newly harvested crude oils from rapeseed oil, soy oil, sunflowerseed oil, maize germ oil, cottonseed oil, palm kernel oil and coconut oil with a free fatty acid content of 2% to 3%. Purified vegetable oils, such as refined or semi-refined vegetable oils of the above-mentioned kind may of course be used as starting materials as well.

Animal crude oils or fats include, for example, milk fat, wool grease, beef tallow, pork lard, fish oils, fish blubber and similar oils and fats. Such animal fats or oils can be used according to the invention in a purified or unpurified state as starting materials for the process according to the invention for the production of biodiesel.

One embodiment of the invention provides for the purification of the starting mixtures containing unpurified free fatty acids prior to the first esterification step. Preliminary purification may be carried out via the purification processes for fats and oils usual for this field, including clarification, filtration, treatment via bleaching clays or treatment via acids or alkali in order to effect separation of inconvenient impurities such as proteins, phosphatides and mucous matter.

According to the invention, during a first step the fat and/or oil containing starting mixtures that were pre-purified or pre-treated, if necessary, are esterified once or several times with a C₁- to C₄-mono alcohol, especially methanol or ethanol, and are transesterified at least twice during a second step with the same C₁- to C₄-mono alcohol. With regard to the present invention, “esterification” is the term used for the reaction of an alcohol with an acid, especially a free fatty acid, leading to the formation of an ester. During the esterification process according to the invention, the free fatty acids of the starting materials are thus converted into the alkyl esters of the free fatty acids. “Transesterification” is the reaction whereby an ester, especially a fatty acid triglyceride, is converted into another ester, especially an alkyl ester of a higher fatty acid, for example via alcoholysis in the presence of acids or alkali.

According to the invention it is especially intended that esterification of the free fatty acids is carried out in the presence of an acid catalyst. “Catalysts” are substances that reduce the activation energy required during the course of a reaction and thus increase the reaction velocity without being present in the end product of the reaction. Acid catalysts are catalysts with a pH value of less than 7. According to the invention, the use of acid, non-volatile esterification catalysts is especially intended, whereby, preferably, heavily volatile inorganic acids such as sulphuric acid, organic derivatives thereof as well as p-toluol sulfonic acid or other suitable substances may be used.

Preferably, the C₁- to C₄-mono alcohol is introduced into the acid esterification process at such a volume whereby a marked excess of mono alcohol is present when compared to the free acids to be esterified, while a clean separation into an oil phase and a mono alhohol phase is also guaranteed at the end of the esterification reaction. Preferably, C₁- to C₄-mono alcohols such as methanol and ethanol are used for the acid esterification of the free fatty acids of the startng material.

According to the invention, esterification of the free fatty acids of the starting material is carried out while using glycerin as a dragging agent. With regard to the present invention, a “dragging agent” is a generally inert substance serving to enrich a component of a composition without being firmly bound within this composition. The dragging agent assists with phase separation by reinforcing the separation effect and serves to set the density of a phase as well as to intercept the reaction water.

According to the invention, glycerine is used as the dragging agent. Glycerine is a component of the fatty acid triglycerides and is released anyway during the subsequent transesterification reactions of the fatty acid triglycerides to alkyl esters. The process according to the invention is thus designed, in a particularly advantageous manner, that the dragging agent glycerine is obtained during the transesterification reactions and is returned to the preceding esterification reactions, so that essentially the dragging agent glycerine needs to be introduced only at the beginning of the process according to the invention.

According to the invention, acid esterification of the free fatty acids is carried out at a temperature of approximately 60° C. to 65° C. at normal pressure. This means that comparatively mild reaction conditions are selected for the acid esterification. The reaction of the acid esterification itself is carried out in one or preferably several stirring vessels or columns in cascade configurations, whereby the cocurrent or countercurrent flow of the two liquid phases passes through the cascade configurations.

In a preferred embodiment of the invention acid esterification of the free fatty acids is carried out in an esterification device designed as a column. “Columns” are column- or tower-shaped reaction vessels, that allow for the separation of at least one phase. The columns used according to the invention may be equipped with suitable column installations such as adjustable or movable column floors, ceramic or metallic packings, fluid distributors, packings consisting of wire fabrics, return flowage distributors and supporting grids. Due to their longitudinal design, the columns utilized according to the invention allow for plug flow as well as a continuously increasing shifting of the equilibrium at certain reactions in the cocurrent or countercurrent flow of two phases. According to the invention it is intended that the flow passes through the esterification columns in a cocurrent or countercurrent flow from the bottom to the top or from the top to the bottom, whereby the columns can be utilized as individual reactors via re-circulation.

According to the characteristics of the fat and/or oil containing starting material used, the fatty acids of the starting mixtures are subjected, according to the invention, to one or several acid esterification steps inside separate esterification columns. Preferably, acid esterification of the initial mixtures is carried out at least twice. In this way an almost 100% esterification of the free fatty acids present in the starting materials is obtained. The individual esterification columns are arranged consecutively to and connected with each other, whereby the esterification mixture contained within one column is conveyed into the consecutive column for further or increased esterification or in order to obtain a reduced acid number. Subsequently or simultaneously a new mixture of dragging agent, mono alcohol and acid catalyst is introduced to the last of the consecutive esterification columns, and subsequently this mixture is used to continue acid esterification of the free fatty acids in the preceding column. The two phases, i.e. the fatty acid mixture as the light phase and the dragging agent loaded methanol-catalyst mixture as the heavy phase, are preferably conveyed in a countercurrent flow. In order to obtain the desired low acid number of the esterification mixture prior to the transesterification reaction, the interconnected columns contain different concentrations of the acid catalyst. It is thus intended according to the invention that the acid catalyst concentrations may be different in the individual esterification columns. For example, if 3 columns are utilized for esterification, the concentrations of the acid catalyst in these three individual columns may be 3% to 10% (in case of high acid numbers of the fatty acid mixture), 1% to 5% (in case of medium-high acid numbers) and 0.1% to 1.5% (in case of low acid numbers, e.g. acid number 10 to 0.5). Since there is the risk that the ester that has formed may hydrolyze again, the concentration of the acid catalyst used is reduced as the fatty acid content is decreasing. Simultaneously, the methanol concentration is increased as the fatty acid concentration is decreasing. This means that in case of a high fatty acid content, esterification is carried out (due to energy-saving reasons during the later separation of the produced reaction water by means of purification via distillation) at a low methanol concentration and a high concentration of the acid catalyst.

According to the invention, the ratio of methanol to fatty acid mixture is increased, especially in the low fatty acid range, by means of a separate cycle restricted to one or two columns for the methanol-catalyst mixture, whereby the ratio of methanol to fatty acid mixture is increased to 2 to 12 times, preferably 4 to 6 times, in relation to the volume of the methanol-catalyst mixture that is used for the fatty acid mixture with a higher acid number in the initial part of acid esterification.

Since the reaction water content of the separate cycle for obtaining a minimum acid number must also be as low as possible (definitely below 1% water), the cycle of methanol-dragging agent-catalyst mixture, increased with regard to volume, is conducted through suitable water retaining devices such as molecular sieves or membrane filters, whereof at least two are provided that are operated in an alternating manner due to the resulting water enrichment.

According to the invention, several or even each of the esterification columns may be operated with such a separate cycle.

According to the invention it is also possible that on completion of the esterification process in one column, only a part of the esterification mixture contained in that column is conveyed to the consecutive column, while an other part of the esterification mixture is conveyed to a preceding column and is re-esterified in that column. This means that if the individual consecutive columns contain different, especially decreasing, concentrations of the acid catalyst, according to the invention that part of the obtained esterification mixture that is conveyed into the consecutive column is further esterified in the presence of a lower concentration of the acid catalyst, while that part of the obtained esterification mixture that is conveyed into the preceding column is esterified in the presence of a higher concentration of the acid catalyst. The solubility of the methanol-catalyst-mixture in the original triglyceride-fatty acid mixture, and thus the reaction velocity, can be improved by means of reintroducing a part of an obtained esterification mixture into a preceding column, and thus overall an almost 100% esterification of the free fatty acids present is obtained.

According to the invention it is intended that the esterification mixture contained in each column after completion of the esterification process is pre-purified prior to being conveyed to the next esterification or transesterification column, to ensure that the subsequent reaction can be carried out under optimum conditions. For example, a part of the produced esterification products, such as reaction water, that may influence the subsequent reaction, can thus be removed. In particular, the esterification mixture obtained in an esterification unit is pre-purified in this way: a mixture comprising the dragging agent, the acid catalyst, the water produced during esterification and the unconverted C₁-C₄-mono alcohol, which constitutes the heavy phase, is at least partially separated via phase separation from the esterification mixture contained in the light phase. The mixture comprising dragging agent, acid catalyst, and mono alcohol, i.e. the heavy phase, separated during preliminary purification via phase separation, is conducted to special devices for the purpose of purification and separation of the components of the mixture. Thus a specific purification and recovery of the components of the mixture, i.e. the dragging agent glycerine, the acid catalyst and the mono alcohol, is carried out according to the invention within the above-mentioned purification devices. The components thus separated and purified can subsequently be reused in process steps according to the invention, particularly in acid esterification process steps.

According to the invention it is especially intended that the heavy phase, including acid catalyst, dragging agent and mono alcohol, is initially conducted into a drying device, wherein most of the unconverted mono alcohol and water are separated from the acid catalyst and the dragging agent. In the drying device, water is either separated via molecular sieves or micro filters, or is evaporated together with the mono alcohol as a water-mono alcohol mixture via distillation. The mono alcohol separated in the drying device is then conveyed to a rectification device for further purification. A “rectification device” is a device for performing a countercurrent or column distillation that serves to decompose liquid or vapor mixtures by conveying the liquid and vapor in a countercurrent flow towards each other while they are in immediate contact with packings, for example. The C₁-C₄-mono alcohol purified in the rectification device has a water content of approximately 1% to 2% and can be recycled from the rectification device into the esterification device and is thus available once again as a reaction ingredient. The rectified methanol that is reused in the esterification device may even have a water content of 1% to 2%, since during the esterification process, water is produced anyway, which is then removed by the dragging agent. Rectification therefore saves energy by saving on reflux ratio.

The mixture containing dragging agent, acid catalyst and trace amounts of mono alcohol, obtained in the drying device, which is almost completely free of water and mono alcohol, is conducted out of the drying device, whereby a part of this mixture can be recycled directly into the esterification columns, while a small partial flow of this mixture is conveyed to the acidification device of the transesterification process. The volume of the partial flow of the dragging agent-catalyst mixture, intended for the acidification device of the transesterification process and relieved of methanol and reaction water, is adjusted precisely to the neutralization of the base catalyst in the soap-containing heavy phase of transesterification and the subsequent sufficient acidification thereof. After acidification has occurred and a sufficient residence time has passed for effecting the separation of fatty acids from the soaps that were partially formed during transesterification, the mixture is conveyed into a separator. The fatty acids obtained are separated in the separator and are returned to the esterification device. For further purification, the remaining mixture, still containing glycerine and considerable trace amounts of C₁- to C₄-mono alcohol, is then conveyed from the separator to a rectification device wherein the remaining mono alcohol is separated and is returned in a purified state to the esterification devices.

One design type of the invention provides that the final esterification column may be designed as an extraction column. The esterification mixture may be extracted in the extraction column while using pure mono alcohol or a mono alcohol-dragging agent mixture, whereby the esterification mixture flowing downstream meets a lighter mono alcohol flowing upstream or a lighter mono alcohol-dragging agent mixture flowing upstream. By means of the extraction the non-esterified free fatty acids are removed and are recycled back to one of the preceding esterification columns for esterification.

The esterification mixture obtained following the last esterification in the last esterification column, or following extraction in the extraction column is characterised by the fact that almost all free fatty acids of the initial fatty acid triglyceride mixture are esterified with the C₁-C₄-mono alcohol. Thus the esterification mixture obtained according to the invention has a particularly low acid number, preferably 0.5 to 1, prior to the transesterification reactions. In addition, the maximum water content of the obtained esterification mixture is 0.5%.

According to the invention it is intended that the esterification mixture obtained after esterification in the last esterification column, with an acid number of 0.5 to 1 and a maximum water content of 0.5%, is conveyed to a transesterification device, arranged consecutively to and connected with the last of the esterification columns, for the base transesterification of the fatty acid glycerides of the starting mixture. According to the invention, base transesterification of the fatty acid triglycerides is carried out with a C₁-C₄-mono alcohol at a temperature of approximately 60° C. to 65° C. under normal pressure, whereby the alkyl esters of the higher fatty acids bound in the fatty acid triglycerides and glycerin are obtained.

According to the invention it is intended that prior to transesterification, one or several fatty acid triglyceride starting mixtures, especially purified fatty acid triglyceride starting mixtures, that do not require acid esterification due to their very low, especially 0% to 1%, free fatty acid content, are added to the esterification mixture. Preferably, the purified starting mixtures added to the esterification mixture are refined or semi-refined products of vegetable oils, such as rapeseed oil, soybean oil, sunflowerseed oil, palm oil, maize germ oil, cotton seed oil, palm kernel oil, coconut oil or similar oils, or refined or semi-refined products from animal fats and oils, such as fish oils and similar oils, or mixtures thereof.

The esterification mixture obtained following the last stage of acid esterification, and the purified starting mixture or mixtures added if necessary, are transesterified according to the invention 2 to 6 times, dependent on the composition of the starting materials, in separate transesterification devices that are arranged consecutively and are connected with each other.

According to the invention, the transesterification devices are designed as columns, with or without packing.

Preferably, the fatty acid triglycerides are transesterified with the same C₁-C₄-mono alcohol as used for the acid esterification of the free fatty acids. Preferably, the mono alcohol used for base transesterification is therefore methanol or ethanol. The base transesterification of the fatty acid triglycerides is preferably carried out at a temperature of 60° C. to 65° C. in the presence of a base catalyst. According to the invention the base catalyst is selected so that optimum transesterification of the fatty acid triglycerides to alkyl ester can take place. Another aspect to be taken into consideration when selecting the base catalyst is that preferably, a salt can form with the base catalyst used, whereby the salt is then used as fertilizer. Preferably the base catalyst used for transesterification of the fatty acid triglycerides is potassium hydroxide. Water-free sodium hydroxide or sodium methylate and other alkaline substances can be used as the base catalyst as well.

The transesterification mixture obtained in a column after transesterification is preferably pre-purified prior to being conveyed to the following column. According to the invention, the transesterification mixture is first subjected to a phase separation in the column, whereby the base catalyst, unconverted mono alcohol and the glycerin formed during transesterification are separated as the heavy phase at least partially from the transesterification mixture present in the light phase. The transesterification mixture purified via phase separation is then further purified in a separator from the column, prior to being conveyed to the consecutive column, with water being the means of purification. Preferably, the water used as a purification agent is pH-conditioned water, or water otherwise buffered in a suitable manner, especially buffered condensation water or buffered softened water. The water used according to the invention must not contain any calcium, otherwise calcium soaps are formed that would block all filtration devices during production and during the later use of the product, due to their amorphous structure. Further amounts of base catalyst, unconverted mono alcohol, soap and glycerine as well as the water used for purification are separated from the transesterification mixture in the separator.

The transesterification mixture thus pre-purified is then conveyed to a consecutive column for further transesterification of the fatty acid triglycerides with mono alcohol, especially if unconverted fatty acid triglycerides are still present in the transesterification mixture. If the purified transesterification mixture is the transesterification mixture obtained in the last of the trans-esterification columns, whereby almost all fatty acid triglycerides have already been transesterified, the transesterification mixture is then purified in at least one additional separator. According to the invention, the transesterification mixture obtained in the last of the columns is purified in at least three separators, arranged consecutively, with suitable water being used as the purification agent.

Subsequently the last transesterification mixture, purified via the separators, is conveyed to a drying device, whereby any remaining water is removed. After the drying process via the drying device the desired end product biodiesel has been obtained in a purified state and can be used directly as fuel.

The heavy phase, obtained in a column via phase separation and separated from the transesterification device, containing unconverted mono alcohol, basis catalyst and glycerine, is conveyed from the column into an acidification device for the purpose of further purification and separation of the components. The watery mixtures of unconverted mono alcohol, base catalyst and glycerine that were obtained in the separators are also conveyed into the above-mentioned acidification device for the purpose of further purification and separation of the components.

In order to saves cost and energy, the small partial flow of the acid catalyst mixture from the acid esterification process is used according to the invention instead of the acid, required by the acidification device, that is usually externally introduced as a new component, whereby the above-mentioned small partial flow has to be diverted anyway for the current minimal renewal of the catalyst-dragging agent mixture.

The dragging agent component contained therein is then returned to the usual glycerine purification process. The purified glycerine is then recycled as the dragging agent to the catalyst cycle of the acid esterification process, which is thus constantly renewed.

The acid catalyst mixture originating from the esterification device is thus united with the alkali heavy phase in the acidification device for the purpose of neutralization and acidification, whereby approximately 1-10% of the total mixture originate from the esterification stage and approximately 90 to 99% originate from the transesterification stage. After acidification has occurred and a sufficient residence time has elapsed so as to effect the separation of the soaps present into fatty acid and salt, the mixture is conveyed from the acidification device to a separator.

The fatty acids released via the soap separation process are separated in the above-mentioned separator in a purified state. The separated fatty acids are then directly returned to the esterification device. The remaining mixture, containing glycerine, the salt being formed from acid catalyst and base catalyst, and C₁-C₄-mono alcohol as well as water, is conveyed from the separator into a rectification device.

The remaining mono alcohol is separated in a purified state from glycerine and the other components in the rectification device and is then recycled for the purpose of acid esterification. The purified mono alcohol returned to the esterification process has a water content of 1% to 2%. The purity of externally introduced fresh methanol is normally above 99.85%, whereby the water content is below 0.1%. Methanol of this quality is used, for example, in the range of the already-low acid numbers of acid esterification, where a water content as low as possible is required. However, this is not the case in the range of the higher acid numbers of acid esterification where larger volumes of reaction water are produced (e.g. 3-6% water, in relation to the fatty acid mixture). According to the invention, for this range methanol may be used that has a considerably higher water content than 0.1%, i.e. 1.0 to 2.0%, as it comes straight from the rectification process. Therefore rectification can be carried out by using a considerably smaller amount of recycled condensed methanol, whereby the essential steam consumption of the total process is thus considerably reduced.

The mixture, which is almost methanol-free obtained simultaneously in the rectification device, comprising glycerine, the salt formed from the esterification and transesterification catalysts as well as reaction water and cleansing water, is conveyed into an evaporation device, where the water in a purified state is separated from the other components, i.e. the so-called crude glycerine consisting of glycerine and salt. The purified water is then evaporated, condensed and returned to the separators, where it can be reused for purification if the transesterification mixtures.

The crude glycerine is then purified via a drying and distillation device, a filtration device and a thin-layer evaporation device, whereby the original mixture comprising acid and base catalyst is separated while a salt suitable for use as fertilizer is being formed. A small partial flow of the newly-obtained glycerine is returned to the esterification devices as a substitute for the diverted dragging agent.

The present invention also concerns a device for producing alkyl esters of higher fatty acids, particularly biodiesel, comprising in an integrated combination an esterification unit with at least two esterification devices for the esterification of free fatty acids, one transesterification unit, arranged consecutively to and connected with the esterification unit, and provided with at least two transesterification devices for the transesterification of the fatty acid triglycerides, a purification unit, arranged consecutively to and connected with the transesterification unit, for purifying the biodiesel produced, and a purification unit arranged consecutively to and connected with the transesterification unit for the purification and separation of the agents utilized in the esterification unit and/or the transesterification unit and/or the purification unit, characterised by the fact that the purification unit is connected with the esterification unit via at least one feeding pipe and at least one discharge pipe, so that the agents utilized in the esterification unit or the transesterification unit can be simultaneously purified and separated in the purification unit and that the agents utilized for esterification can be recycled to the esterification unit.

The device according to the invention for producing biodiesel is suitable, in a particularly advantageous manner, for the production of biodiesel from new and used vegetable, animal or industrial oils or fats. The device according to the invention is provided in particular with an esterification unit and a transesterification unit arranged consecutively to and connected with the esterification unit, so that the products formed in the esterification unit are conveyed to the transesterification unit where they are subjected to transesterification.

The esterification unit according to the invention is provided in an advantageous manner with several esterification devices designed especially as columns, as well as several transesterification devices also designed especially as columns, whereby the esterification unit is equipped with at least two and up to e.g. seven separate columns and whereby the transesterification unit is equipped with at least two and up to e.g. four separate columns. The device according to the invention allows the variable utilization of the individual esterification and/or transesterification columns. The number of columns actually used when the device according to the invention is being operated is dependent on the consistency, i.e. composition and purity, of the fat or oil containing starting mixtures and on the desired composition and purity of the end product. If, for example, the initial mixtures used do not contain any free fatty acids, or only negligible trace amounts thereof, the entire esterification unit, i.e. the all columns of the esterification unit, can be separated from the other units of the device according to the invention and can be deactivated, so that the initial mixtures are only subjected to transesterification. If the initial mixture used contains mainly free fatty acids but no fatty acid triglycerides or only negligible trace amounts thereof, the entire transesterification unit can be separated from the esterification unit according to the invention and may be deactivated, for example, so that only esterification reactions are carried out. Naturally, individual columns of the esterification unit and/or the transesterification unit may be deactivated as well. Thus it is possible to utilize only two columns per unit, for example.

In addition, the device according to the invention for producing biodiesel is equipped with two purification units, whereby one of the purification units (designated as biodiesel purification unit in the text below) serves to purify and obtain the product, i.e. biodiesel, while the second purification unit (designated as agent purification unit in the text below) serves to purify, separate and recover the agents that were used in the esterification unit, the transesterification unit and the biodiesel purification unit. The biodiesel purification unit is connected especially with the transesterification unit, so that the products formed in the transesterification unit, i.e. the biodiesel, can be purified. In addition, the biodiesel purification unit is connected with the agent purification unit, so that the mixtures obtained in the biodiesel purification unit, which include the agents used in the esterification and transesterification units as well as the agents used in the biodiesel purification unit, can also be treated and obtained in the agent purification unit. The agents utilized in the esterification unit are especially a C₁- to C₄-mono alcohol, an acid catalyst and a dragging agent. The agents utilized in the transesterification unit are a base catalyst and C₁- to C₄-mono alcohol. The agent utilized in the biodiesel purification unit is in particular water.

The device according to the invention for producing biodiesel is especially characterised by the fact that the agent purification unit is connected via feeding pipes with the transesterification unit as well as with the esterification unit. The mixtures formed in the esterification unit and in the transesterification unit, respectively, which are separated via phase separation from the respective product mixtures in these units, and which include the agents and by-products used in these units, can be conveyed via the feeding pipes directly to the agent purification unit where they are purified and separated.

The device according to the invention thus allows in an advantageous manner to simultaneously purify the agents used for carrying out esterification reactions, transesterification reactions and biodiesel purification within the same purification unit. The result is that due to the considerably reduced expenditure with regard to the equipment required, the device according to the invention is far more cost-effective, i.e. considerably less expensive, than other devices for producing biodiesel that are already known according to the state-of-the-art technology. For example, the esterification unit does not need to be combined with an additional purification unit. Producing biodiesel by means of the device according to the invention therefore considerably reduces production costs.

The agent purification unit is connected with the esterification unit in an advantageous manner via outlet conduits, whereby the agents that are separated, purified and thus recovered in this purification unit are directly returned via cycle systems to the esterification unit where they can be reused. The device according to the invention for producing biodiesel is thus provided, according to the invention, with several integrated cycles serving especially to purify, separate and recover the agents used for carrying out esterification reactions, transesterification reactions and/or purification of the reaction mixtures and to return these agents to the esterification unit in particular, whereby the individual cycles for the respective agents to be purified are partially combined and partially separate. These cycles, integrated in the device according to the invention, permit the almost complete recovery and reuse, i.e. recycling, of the agents used.

The esterification unit according to the invention includes one or several esterification devices. For a preferred design type of the invention, the esterification unit includes 2 to 8 esterification devices, arranged consecutively to and connected with each other. Preferably the esterification devices are designed as columns. The columns according to the invention may be provided with suitable column installations such as adjustable or movable column plates, packings, fluid distributors, sorted packing consisting of wire fabrics, reflux distributors and supporting grids. According to the invention the last of the columns may be designed as an extraction column.

The separate esterification devices are connected with each other via feeding pipes, whereby any one esterification device is connected to a consecutive esterification device, so that the reaction product or esterification mixture formed in the device can be conveyed to the consecutive device as the light phase. According to the invention, the respective feeding pipes serving to connect the consecutive esterification devices are each provided with subsidiary pipes for the purpose of connecting an esterification device with a preceding one. These subsidiary pipes thus serve to convey a partial flow of the product mixture formed in an esterification device to a preceding esterification device or once again into the same esterification device, in an advantageous manner.

According to the invention at least one esterification is additionally connected via at least one feeding line with a mixing device, so that a mixture of C₁- to C₄-mono alcohol, acid catalyst and dragging agent that is produced in the mixing device can be conducted to the respective esterification device.

A further embodiment of the invention provides that two or more or each of the esterification devices are connected via separate feedings with one or several mixing devices. The individual esterification devices may each be connected to a separate mixing device or may be connected to the same mixing devices. In this way, different or identical mixtures containing identical or different concentrations of mono alcohol, acid catalyst and dragging agent can be conducted to the individual esterification devices.

A further embodiment of the invention provides that the esterification devices are each interconnected via an additional feeding. At least a partial flow of the heavy phase obtained in a column via phase separation after esterification and containing unconverted glycerin, unconverted mono alcohol as well as unconverted acid catalyst can be conveyed to the preceding column via the above-mentioned additional feeding.

The additional feeding may be connected according to the invention with a drying device wherein the reaction water and/or monoalcohol are removed from the heavy phase. These drying devices may be designed as a distillation device for the evaporation of a water-mono alcohol mixture, for example, or as a molecular sieve or micro filter, respectively, in order to remove the water as a permeate. Subsequently the heavy phase that is now free of water and/or mono alcohol is conducted into the preceding column.

A preferred embodiment of the device according to the invention is thus conceptualized in such a way that on completion of the esterification process in a column, the unconverted acid catalyst, the unconverted glycerine and the unconverted monoalcohol are each conveyed into a preceding column, while the esterification mixture formed in the column is conveyed into the respective consecutive column. Thus the acid catalyst, glycerine and mono alcohol are conveyed from column to column towards the esterification mixtures in a countercurrent flow. Due to this fact as well as the fact that according to the invention several mixtures comprising acid catalyst, glycerine and mono alcohol can be introduced separately into the esterification unit, whereby the concentration of the components of these mixtures may differ or may be identical, it is possible to use different concentrations of acid catalyst, glycerine and mono alcohol for each of the individual columns. In this way it is possible according to the invention to increase the mono alcohol content as the fatty acid content is decreasing, and to reduce the concentration of the acid catalyst.

According to the invention it is further intended that the last esterification device of the esterification unit is connected via at least one inlet conduit with the consecutive transesterification unit, so that the products formed in the esterification unit, especially in the last esterification device, can be conveyed to the transesterification unit.

In a preferred embodiment, the transesterification unit of the device according to the invention includes at least 2 and not more than 6 transesterification devices, arranged consecutively to and connected with each other. According to the invention the transesterification devices are designed as columns as well. Each transesterification column is connected to a mixing device via at least one feeding whereby a mixture formed in the mixing device, comprising base catalyst and C₁-C₄-mono alcohol, can be supplied to the transesterification column. In addition, each of the transesterification devices is connected via a separate feeding each to the agent purification device, especially to an acidification device of the agent purification device. A mixture that is separated from the transesterification mixture formed in each transesterification device via phase separation, whereby this mixture includes in particular base catalyst, soaps, excess mono alcohol and glycerine formed during transesterification, is conveyed via the feeding pipe directly to the acidification device for the purpose of purifying the above-mentioned compounds. In addition, each transesterification device is connected via at least one feeding to the biodiesel purification unit, especially to a separator of the biodiesel purification unit, so that following partial purification via phase separation, the transesterification mixture that was formed in a transesterification device can be conveyed to a separator of the biodiesel purification unit where it is further purified. After purification, the now purified conversion mixture is conveyed via a feeding located between the separator and the consecutive transesterification device into the next transesterification device where it is once again transesterified. The individual transesterification devices of the transesterification unit are thus connected with each other via one respective separator of the biodiesel purification unit. The last transesterification device is also connected via a feeding to a separator of the biodiesel purification unit. However, this separator is provided with an inlet conduit to another separator arranged consecutively to the former, so that the transesterification mixture formed in the last transesterification unit, i.e. biodiesel, can be further purified. The last transesterification mixture is purified via at least three additional separators, whereby these additional separators are each connected via a feeding to the separators of the biodiesel purification unit.

In a preferred embodiment of the invention the biodiesel purification unit consists of at least 4 separators, arranged consecutively to and connected with each other, as well as a drying device. The transesterification mixtures are separated in the separators by means of water from the base catalyst, excess mono alcohol, glycerine formed during transesterification and trace amounts of acid catalysts. According to the invention, the separators are connected to the drying device of the biodiesel purification unit via at least one feeding pipe, whereby the product mixture separated from base catalyst, mono alcohol, acid catalyst and dragging agent in the separators, i.e. biodiesel, is conducted into the drying device. The purified end product biodiesel is then obtained in the drying device.

According to the invention, the biodiesel purification unit is connected via special feedings with the agent purification unit for purifying the agents used. In particular, the separators of the biodiesel purification unit are connected via an inlet conduit with the acidification device of the agent purification unit, in order to conduct the mixture comprising base catalyst, mono alcohol, acid catalyst and dragging agent that has been separated in the separators into the agent purification unit, in particular into the acidification device thereof.

According to the invention, the agent purification unit serving to purify the agents used in the esterification unit, the transesterification unit and the biodiesel purification unit, is equipped with at least one drying device, one acidification device, a further separator, a rectification device, an evaporation device, a distillation device, a membrane evaporation device and a filtration device.

The agent purification unit is connected in an advantageous manner not only with the transesterification unit and the biodiesel purification unit, but via at least one feeding with the esterification unit as well. The esterification unit is connected via a feeding, for example, with the drying device of the of the agent purification unit, whereby the drying device is also connected via another feeding to the acidification device of the agent purification unit. The watery mixtures comprising unconverted C₁- to C₄-mono alcohol, acid catalyst, dragging agent and the water produced during esterification, obtained in the esterification unit via phase separation, are conducted via the above-mentioned feeding into the drying device of the agent purification unit. The mixtures obtained in the esterification unit are further purified in the drying device, whereby the C₁- to C₄-monoalcohol in particular is separated from the remaining components of the mixtures and is conveyed via a feeding from the drying device into the consecutive rectification device of the agent purification unit. In addition, the drying unit is connected via a further feeding with the acidification unit, in order to conduct a small partial flow of the mixture comprising acid catalyst, dragging agent and remaining trace amounts of the C₁- to C₄-mono alcohol, separated in the drying device, to the acidification device.

The acidification device is also directly connected via special feeding to the transesterification units and the separators of the biodiesel purification unit. These special feedings serve to conduct the mixtures formed via phase separation in the transesterification devices and the mixtures formed in the separators during biodiesel purification, whereby both of these mixtures comprises water, glycerine, soaps, base catalyst and excess mono alcohol not converted during transesterification, into the acidification device.

The acidification device is connected via a feeding to the separator of the agent purification unit. Separation of the fatty acids released from the soap content takes place in this separator, and subsequently the separated fatty acids are conducted out of the separator via a further feeding and are returned to the esterification unit. The separator is further connected via a feeding with the rectification device of the agent purification unit, in order to convey the watery mixture formed in the separator, comprising acid catalyst, base catalyst and considerable amounts of mono alcohol into the rectification device.

Separation of the mono alcohol from the watery mixture as well as further purification is carried out in the rectification device. The mono alcohol purified in the rectification device is re-conveyed via a discharge connecting the rectification device with the esterification unit into the latter and can thus be reused in the esterification unit. The rectification device is further connected via a feeding with the evaporation device, in order to convey the mixture separated in the rectification device from mono alcohol and comprising water, trace amounts of glycerine as well as base and acid catalyst, into the evaporation device for further purification.

Water is separated from the conveyed mixture within the evaporation device. The evaporation device is provided with a feeding for condensation, whereby the water separated in the evaporation device is condensed and is then conveyed to the biodiesel purification unit where it can be reused, particularly in the separators of the biodiesel purification unit. The evaporation device is connected via a further feeding with the drying device of the agent purification unit and distillation device, in order to further purify the crude glycerine now devoid of water, the mixture of glycerine, the acid catalyst and the base catalyst and the remaining traces of water.

Separation of glycerine from all accompanying substances, mainly the salt formed from the acid and base catalyst, is carried out in the distillation device. The salt is conveyed out of the distillation device via a feeding connecting the distillation device with a membrane evaporation device, and the still-present accompanying glycerine is recovered.

The salt is suitable for use as fertilizer. The distillation device is connected via a pipe with a filtration device. Pure glycerine is conducted into the filtration device via the pipe connecting the distillation device with the filtration device, wherein it is further purified to pharmaceutical glycerin which can fulfil the highest requirements.

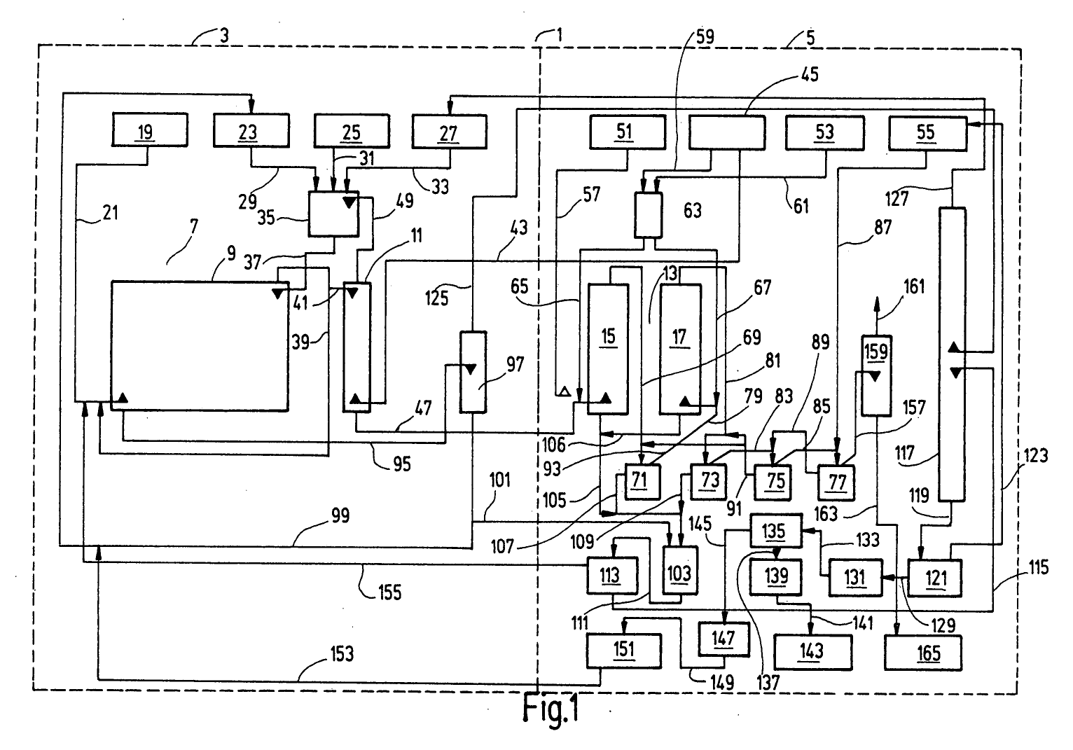

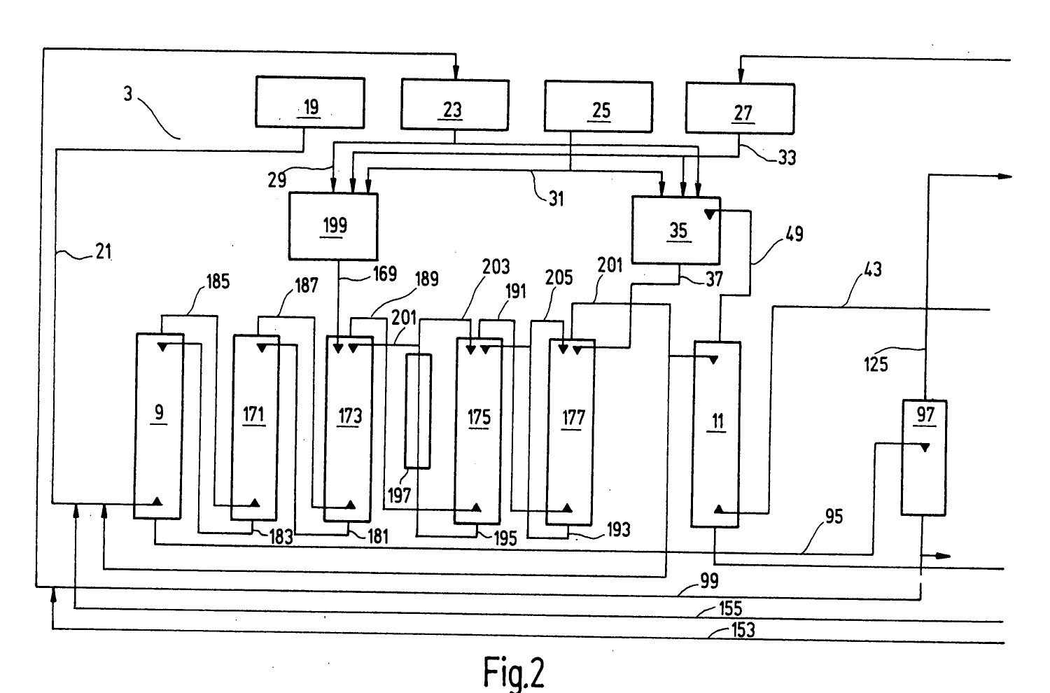

The present invention is further explained by means of the following FIGS. 1 and 2.

In FIG. 1 a device according to the invention for producing biodiesel, including an esterification unit, a transesterification unit, a purification unit for purifying the produced biodiesel and a purification unit for purifying and separating the agents used in the esterification unit and in the transesterification unit is shown schematically.

Another design type of the esterification unit is shown schematically in FIG. 2.

FIG. 1 shows a device 1 according to the invention for producing biodiesel from oil and/or fat containing starting mixtures containing free fatty acids, equipped with an integrated biodiesel purification unit 6 and an integrated agent purification unit 8. The device 1 includes an esterification unit 3 with two esterification devices 9 and 11 designed as columns. The esterification unit 9 is connected via a pipe 37 with a mixing device 35. Pipe 41 passes from column 9 into the esterification column (or extraction column) 11 in order to conduct the esterification mixture produced in column 9 into column 11. The esterification column 11 is further connected via a pipe 43 with a storage container 45 for the C₁-C₄-mono alcohol, so that a new batch of mono alcohol can be introduced into column 11. Pipe 47 passes from column 11 into the transesterification unit 5 that is provided with two transesterification devices 15 and 17 designed as columns.

The transesterification column 15 and the transesterification column 17 are each connected via pipes 65 and 67 with the mixing device 63, whereby the latter is also connected via conduit 59 with the storage container 45 for the mono alcohol and via pipe 61 with the storage container 53 for the base catalyst. The column 15 is further connected via pipe 57 with the storage container 51 for a second starting mixture having an extremely small free fatty acid content. The transesterification mixture produced in column 15 is subjected to phase separation, whereby the watery mixture separated from the transesterification mixture, comprising the mostly unconverted mono alcohol, glycerine and the base catalyst, is conducted from column 15 via pipe 105 into the acidification device 103. The transesterification mixture produced in the transesterification column 15 and pre-purified via phase separation, is conveyed from column 15 via the pipe 69 into separator 71 for further purification.

The separator 71 is supplied with water from the water storage container 55 via pipe 93. The transesterification mixture produced in column 15 is further separated in separator 71 from dispersed or dissolved glycerine as well as from base catalyst and C₁-C₄-mono alcohol which is conveyed via pipe 107 into the acidification device 103. The transesterification mixture from column 15 that has been purified in separator 71 is conveyed via pipe 79 into the second transesterification column 17.

Phase separation takes place in the transesterification column 17 as well, whereby the mixture comprising mostly glycerine, base catalyst and mono alcohol that was separated via phase separation is conveyed via pipe 106 into pipe 105 and then into the acidification device 103. The transesterification mixture produced in esterification column 17, pre-purified via phase separation, is conveyed via pipe 81 into the separator 73 for further purification, whereby the watery mixture separated in the separator 73, comprising glycerine, acid and base catalyst as well as unconverted C₁-C₄-mono alcohol, is conducted from the separator 73 via pipe 109 into pipe 105 and then into the acidification device 103. The separator 73 is supplied with water or condensed steam, respectively, via feeding 91 that is connected with the separation device 75. The transesterification mixture from the transesterification column 17, purified in separator 73, is conducted via pipe 83 into the separator 75, whereby the latter is supplied with water or condensed steam, respectively, via pipe 89 which is also connected with the separator 77. The transesterification mixture further purified in separator 75 is then conducted via pipe 85 into the separator 77, which is supplied with water from the water storage container 55 via pipe 87.

When purification of the transesterification mixture is completed, the mixture is conveyed via pipe 157 into the drying device 159. The water produced in the drying device 159 during the drying process of the purified transesterification mixture is drained via discharge 161, while the purified and concentrated end products thus obtained are conveyed from the drying device 159 via pipe 163 into the storage container 165 for the desired end product.

The mixture comprising glycerine and the acid and base catalyst as well as unconverted C₁-C₄-mono alcohol, conveyed in the acidification device 103, is conducted via pipe 101 into the separator 113 for the separation of the fatty acids, whereby the separated fatty acid is conducted via pipe 155 to pipe 21 and then returned into the esterification unit 3. The mixture formed after separation of the fatty acid in the separator 113 is conducted via pipe 115 into a rectification device 117.

Unconverted mono alcohol, produced in the drying device 97, is also supplied to the rectification device 117 via pipe 125 from the esterification unit 3. The C₁-C₄-mono alcohol is then further purified in the rectification device 117. The mixture produced in the rectification device 117 after separation of the mono alcohol is conveyed via pipe 119 into the evaporation device 121, wherein the water added to the separators is evaporated from the water-containing mixture, is then condensed and recovered, and is conveyed via pipe 123 back to the water storage container 55. The remaining components of the mixture produced in the evaporation device 121 are conveyed via pipe 129 into the drying device 131 and then via conduit 193 into the distillation device 135. Glycerine is separated in the distillation device from the salt produced from the acid and base catalyst and is conducted via pipe 145 to the filtration device 147 for further purification. A partial flow of the glycerine that has been purified by means of filtration is conducted via pipe 149 into a glycerine storage container 151 and then via pipe 153 to pipe 99, which serves to connect the drying device 97 with the dragging agent storage container, i.e. the glycerine storage container 23. In other words, the glycerine purified in the filtration device is thus conducted into the dragging agent storage container 23, whereby the dragging agent glycerine required in the esterification unit 3 is taken from the glycerine storage container 23.

The device according to the invention for producing biodiesel is therefore designed in such a way that the C₁- to C₄-mono alcohol that was not converted in the esterification unit is conducted out of the actual esterification devices and is conveyed into purification devices, whereby the unconverted mono alcohol that was conducted out of the transesterification unit is conveyed into the above-mentioned purification devices as well. The unconverted mono alcohol is purified in the purification device and is then transferred to the mono alcohol storage container, whereby the mono alcohol required in the esterification unit for the purpose of esterification is taken from the mono alcohol storage container. The dragging agent required in the esterification unit is conducted into the same purification devices as the unconverted mono alcohol and is then transferred to the dragging agent storage container, i.e. the glycerine storage container, and is thus available for acid esterification in the esterification unit. The catalysts used for acid and base catalyzing processes, if they were not already recycled into preceding columns via internal cycles, are conducted by way of the same purification devices. After having been purified, the acid and the base catalyst are subjected to a reaction whereby a salt suitable as fertilizer is obtained.

FIG. 2 shows a further design type of the esterification unit 3 with the esterification devices 9, 171, 173, 175, 177 and 11, designed as columns. The starting mixture that is to be esterified is introduced into the first esterification column 9. On completion of the esterification process in column 9 the esterification mixture produced in column 9 is conveyed as the light phase via pipe 185 to the consecutive esterification column 171 for further esterification. The esterification mixture produced following esterification in column 171 is then conducted via pipe 187 into the consecutive esterification column 173 for further esterification, then via pipe 189 into the consecutive column 175, then via pipe 191 into the consecutive column 177 and is finally conducted via pipe 201 into the last column 11.

According to the invention the esterification unit 3 is supplied by the two separate mixing devices 199 and 35 with mixtures of the agents required for esterification, i.e. acid catalyst, mono alcohol and glycerine. The mixing devices 199 and 35 are connected via pipe 29 with the glycerine storage container 23, via pipe 31 with the storage container 25 for the acid catalyst, and via pipe 33 with the storage container 27 for used mono alcohol, and are supplied from storage containers 23, 25 and 27 with glycerine, acid catalyst and mono alcohol. The mixtures produced in the mixing devices 199 and 35, containing acid catalyst, glycerine and mono alcohol, may vary with regard to the concentrations of the components.

The mixture containing acid catalyst, glycerine and mono alcohol is directly introduced into column 173 via pipe 169, which serves to connect the mixing device 199 with the column 173. On completion of the esterification process in column 173, the heavy phase containing unconverted glycerine, unconverted mono alcohol and unconverted acid catalyst is separated via phase separation from the light phase containing the esterification mixture, and is conveyed via pipe 181 into the preceding column 171. On completion of the esterification process, a phase separation is carried out in column 171 as well, whereby the obtained heavy phase is conveyed via pipe 183 into the first column 9.

The mixture comprising glycerine, catalyst and mono alcohol produced in the mixing device 35 is introduced directly into the esterification column 177 via pipe 37. On completion of the esterification process a phase separation is carried out in column 177 as well, whereby a partial flow of the produced heavy phase is conducted into the preceding column 175 via pipe 193, and a partial flow of the heavy phase is re-conducted to column 177. The heavy phase obtained in column 175 on completion of esterification and phase separation is then conveyed via pipe 195 into a drying device 197 for the separation of the reaction water and is then re-conducted via pipe 201 into column 175, whereby a partial flow of the purified heavy phase is conveyed into the preceding column 173.

According to the invention it is therefore intended that on completion of the esterification process in a column, the unconverted acid catalyst, unconverted glycerine and unconverted mono alcohol are conducted into the respective preceding column, while the esterification mixture produced in the column is conducted to the respective consecutive column.

Acid catalyst, glycerine and mono alcohol are thus conducted from one column to the next column in a countercurrent direction to the esterification mixtures. This, as well as the fact that according to the invention two mixtures comprising acid catalyst, glycerine and mono alcohol are introduced separately into the esterification unit, whereby the concentration of the components of the above-mentioned mixtures may differ, but may also be identical, allows that different concentrations of acid catalyst, glycerine and mono alcohol can be used in the individual columns. Thus it is possible according to the invention to increase the mono alcohol content and to reduce the concentration of the acid catalyst with decreasing fatty acid content.

1. A non-pressurized method for the continuous production of alkyl esters of higher fatty acids from fatty acid triglyceride starting mixtures containing free fatty acids, including an integrated combination of acid esterification and basic transesterification, said method comprising:
a) at least one esterification of the free fatty acids in separate esterification devices connected with each other, with a C₁- to C₄-mono alcohol in the presence of an acid catalyst and glycerine as a dragging agent, at 60° C. to 65° C. to produce an esterification mixture is produced,
b) partial purification of the first esterification mixture via partial separation of the dragging agent, acid catalyst and unconverted C₁- to C₄-mono alcohol,
c) transesterification of the fatty acid triglycerides, carried out at least twice, in separate transesterification devices connected with each other, with a C₁- to C₄-mono alcohol in the presence of a base catalyst at 60° C. to 65° C. to produce a transesterification mixture and
d) purification of the transesterification mixture via separation of the basic catalyst, unconverted C₁- to C₄-monoalcohol and the glycerine produced during transesterification, by a treatment with water in at least one separator with subsequent drying, wherein the C₁- to C₄-mono alcohol used for esterification, the conveying agent glycerin used for esterification and the water used for purification of the transesterification mixture are at least partially recovered from the esterification and transesterification mixtures and further wherein after purification the acid and base catalysts from the esterification and transesterification mixtures are converted, resulting in the production of a salt suitable for use as a fertilizer.

2. The method according to claim 1, wherein the fatty acid triglyceride initial mixtures containing free fatty acids are selected from the group consisting of used and unused, unpurified and purified vegetable, animal and industrial oils and fats and mixtures thereof with a free fatty acid content of 0% to 100%.

3. The method according to claim 1 wherein the unpurified oils and fats are selected from the group consisting of soapstock, brown grease, yellow grease, industrial tallow, industrial lard, oil used for deep-frying, animal fat waste products, edible tallow, unpurified crude vegetable oils, unpurified animal fats and mixtures thereof.

4. The method according to claim 3, wherein the unpurified crude vegetable oils are selected from the group consisting of rapeseed oil, soybean oil, sunflowerseed oil, palm oil, maize germ oil, cotton seed oil, palm kernel oil and coconut oil.

5. The method according to claim 2 wherein the unpurified starting mixtures are purified prior to esterification.

6. The method according to claim 2, wherein the purified oils or fats are refined or semi-refined products of vegetable or animal oils or fats.

7. The method according to claim 6, wherein the vegetable or animal oils or fats are selected from the group consisting of rapeseed oil, soybean oil, sunflowerseed oil, palm oil, maize germ oil, cotton seed oil, palm kernel oil and coconut oil.

8. The method according to claim 1, wherein the esterification device is a column with or without ceramic or metallic packings or packings made of wire fabric.

9. The method according to claim 1, wherein the C₁- to C₄-mono alcohol used for esterification is methanol or ethanol.

10. The method according to claim 1, wherein the acid catalyst used for esterification is sulphuric acid or p-toluol sulfonic acid.

11. The method according to claim 1, wherein the free fatty acids are esterified 2 to 8 times in separate columns consecutive to and connected with each other, depending on the fatty acid content of the starting mixtures.

12. The method according to claim 11, wherein the esterification mixture obtained from one column is conducted from that column into the consecutive column and whereby, after the addition of a mixture containing a dragging agent, a mono alcohol and an acid catalyst, the esterification mixture is esterified again.

13. The method according to claim 12, wherein different amounts of acid catalyst are introduced into the separate columns.

14. The method according to claim 12 wherein a part of the esterification mixture from one column is conducted to the consecutive column and whereby a part of the resulting esterification mixture is re-conducted into the preceding column.

15. The method according to one of claim 11 wherein the esterification mixture is partially purified prior to being conveyed into the consecutive column.

16. The method according to claim 15, wherein a mixture containing dragging agent, acid catalyst, unconverted mono alcohol and water produced during esterification is separated as the heavy phase via phase separation from the esterification mixtures and is conveyed to devices for the purification and separation of the components of the mixture.

17. The method according to claim 16, wherein the heavy phase is conducted into a drying device for the separation of mono alcohol and water.

18. The method according to claim 17, wherein water is separated via molecular sieves or micro filters within the drying device or wherein a mono alcohol and water mixture is evaporated via distillation.

19. The method according to claim 17 wherein at least one of the mono alcohol and the mono alcohol-water mixture is conducted from the drying device into a rectification device for further purification.

20. The method according to claim 19, wherein the mono alcohol purified in the rectification device, having a water content of approximately 1% to 2% suitable for use in columns due to a higher fatty acid content, is re-conducted from the rectification device into the esterification device.

21. The method according to claim 17 wherein a partial flow of the mixture obtained in the drying device after separation of mono alcohol and water and containing dragging agent and acid catalyst, is re-conducted from the drying device into the esterification device, and wherein a partial flow of the above-mentioned mixture is conducted into an acidification device.

22. The method according to claim 8 wherein the esterification mixture obtained after esterification in the last esterification column is conducted into an extraction column and is subjected to extraction in that column utilizing pure mono alcohol or a mono alcohol-dragging agent mixture for the removal of non-esterified free fatty acid.

23. The method according to claim 8, wherein an esterification mixture is obtained on completion of the last esterification step in the last esterification column or on completion of the extraction step in the extraction column and whereby almost all free fatty acids have been esterified and whereby the above-mentioned mixture has an acid number of approximately 1 to 0.5 and a maximum water content of 0.5%.

24. The method according to claim 23, wherein the esterification mixture is conducted into a transesterification device connected to the last esterification column or to the extraction column for the basic transesterification of the fatty acid glycerides.

25. The method according to claim 24, wherein prior to transesterification one or several fatty acid triglyceride starting mixtures with a free fatty acid content of 0% to 1% are added to the esterification mixture.

26. The method according to claim 25, wherein the starting mixture added to the esterification mixture is a refined or semi-refined product of a material selected from the group consisting of rapeseed oil, soybean oil, sunflowerseed oil, palm oil, maize germ oil, cotton seed oil, palm kernel oil and coconut oil and mixtures thereof.

27. The method according to claim 24, wherein the esterification mixture to be transesterified and the starting mixture that was added if required are transesterified 2 to 6 times depending on the composition of the total mixture in separate transesterification devices that are arranged consecutively to each other and are connected with each other.

28. The method according to claim 27, wherein the transesterification devices are columns with or without ceramic or metallic packings or packings made of wire fabric.

29. The method according to claim 24 wherein the C₁- to C₄-mono alcohol used for transesterification is methanol or ethanol.

30. The method according to claim 24 the base catalyst used for transesterification is selected from the group consisting of potassium hydroxide, sodium hydroxide and sodium methylate.

31. The method according to claim 24 wherein the transesterification mixture obtained in a column is subjected to preliminary purification.

32. The method according to claim 31, wherein a mixture of mono alcohol, base catalyst and glycerine is separated as the heavy phase via phase separation from the transesterification mixture and is conducted for further purification and separation of the components to an acidification device and subsequently to a separator arranged consecutively to the acidification device.

33. The method according to claim 21 wherein the heavy phase separated from the transesterification mixture is mixed in the acidification device with the partial flow of the dragging agent and acid catalyst mixture that was separated following esterification and conducted into the acidification device, wherein the partial flow is proportioned in such a way that the base catalyst of the heavy phase is neutralised and the heavy phase is sufficiently acidified.

34. The method according to claim 31, wherein the pre-purified transesterification mixture is conducted into a separator for further purification.

35. The method according to claim 34, wherein a water-containing mixture comprising mono alcohol, soap, base catalyst and glycerine is separated in the separator from the transesterification mixture with water.

36. The method according to claim 35, wherein pH-conditioned water or buffered water is used.

37. The method according to claim 34 wherein the purified transesterification mixture is conducted from the separator to a consecutive transesterification column for further transesterification.

38. The method according to of the claim 34 wherein the purified transesterification mixture of the last transesterification column is conducted out of the separator into at least one additional separator for further purification.

39. The method according to claim 38, whereby wherein the transesterification mixture from the at least one additional separator is conducted into a drying device where it is dried and purified.

40. The method according to claim 39, wherein following purification in the drying device a product mixture corresponding to biodiesel and comprising mainly C₁- to C₄-alkyl esters of the free fatty acids and C₁- to C₄-alkyl esters of the higher fatty acids is obtained from the fatty acid triglycerides.

41. The method according to claim 34 wherein the water-containing mixture of mono alcohol, soap, base catalyst and glycerine separated from the transesterification mixture in the separator is conducted into the acidification device and then into a consecutive separator.

42. The method according to claim 32 wherein the fatty acids formed during transesterification are partially separated from the remaining components of the water-containing mixture in the separator that is consecutive to the acidification device and are re-conducted into the esterification device.

43. The method according to claim 42, wherein the remaining components of the water-containing mixture are conducted from the separator into the rectification device.

44. The method according to claim 43, wherein the mono alcohol is separated in the rectification device from the remaining components of the water-containing mixture and is re-conducted to the esterification device in a purified state, wherein the water content of the purified mono alcohol is approximately 1% to 2%.

45. The method according to claim 44, wherein the remaining components of the water-containing mixture are conducted from the rectification device into an evaporation device.

46. The method according to claim 45, wherein the water is separated in the evaporation device and is re-conducted into the separators.

47. The method according to claim 46, wherein the mixture comprising glycerine, acid catalyst and base catalyst is conducted into a distillation device for further purification.

48. The method according to claim 47, wherein the glycerine is separated in the distillation device from the catalysts and is partially re-conducted into the esterification device in a purified state after filtration via a filtration device.

49. The method according to claim 47, wherein acid and base catalyst are conducted from the distillation device into a thin-layer-evaporation device where the acid and base catalysts are converted, resulting in the formation of a salt suitable as a fertilizer.

50. An apparatus for the production of biodiesel from fatty acid triglyceride starting mixtures containing free fatty acids, said apparatus comprising, in an integrated combination, an esterification unit (3) having at least two esterification apparatuses (9, 171, 173, 175, 177, 11) for the esterification of the free fatty acids, a downstream transesterification unit (5) connected to the esterification unit and having at least two transesterification apparatuses (15, 17) for the transesterification of the fatty acid triglycerides, a downstream purification unit (6) connected to the transesterification unit (5) and intended for the purification of the biodiesel, and a downstream purification unit (8) connected to the transesterification unit (5) and intended for the purification and separation of compositions used in at least one of the esterification unit (3) the transesterification unit (5) and for the purification and removal of the water used in the purification unit (6), wherein the purification unit (8) has at least one evaporation apparatus (121) for the recovery of water and is connected by at least one feed line (101) and at least one discharge line (127, 153, 155) to the esterification unit (3), so that the compositions used in the esterification unit (3) and the transesterification unit (5) are simultaneously purified and separated in the purification unit (8) and compositions used for the esterification are recycled to the esterification unit (3).

51. The apparatus as claimed in claim 50, wherein the compositions used in the esterification unit (3) are a C₁-C₄-monoalcohol, an acidic catalyst and an entraining agent, and the compositions used in the transesterification unit (5) are a basic catalyst and the C₁-C₄-monoalcohol.

52. The device according to claim 50, wherein the esterification unit (3) is provided with 2 to 8 consecutive esterification devices (9, 171, 173, 175, 179, 11) that are connected to each other.

53. The device according to claim 50, wherein the esterification devices (9, 171, 173, 175, 179, 11) are designed as columns.

54. The device according to claim 53, wherein a last column (11) is an extraction column.

55. The device according to claim 50, wherein the esterification devices (9, 171, 173, 175, 179, 11) are connected via one inlet conduit (41, 185, 187, 189, 191, 201) each for conveying the products formed in one device as a light phase into the consecutive device.

56. The device according to claim 55, wherein the feeding pipes (41, 185, 187, 189, 191, 201) are provided with a subsidiary pipe (39) for re-conducting a partial flow of the products formed in the esterification devices (9, 171, 173, 175, 179, 11) into the same or a preceding esterification device.

57. The device according to claim 50, whereby wherein at least one esterification device (9, 171, 173, 175, 179, 11) is connected to a mixing device (35, 199) via afeeding (37, 169) for conducting a mixture of C₁- to C₄-mono alcohol, acid catalyst and dragging agent produced in the mixing device (35, 199) into the esterification device (9, 171, 173, 175, 179, 11).

58. The device according to claim 57, wherein at least two of the esterification devices (9, 171, 173, 175, 179, 11) are each connected to one separate mixing device (35, 199) via separate feedings (37, 169) for conducting mixtures with identical or different concentrations of C₁- to C₄-mono alcohol, acid catalyst and dragging agent into the esterification devices (9, 171, 173, 175, 179, 11).

59. The device according to claim 50, wherein the esterification devices (171, 173, 175, 179, 11) are connected via one feeding (181, 183, 193, 195) each for conveying at least a partial flow of the heavy phase, obtained via phase separation after esterification in an esterification device and containing unconverted glycerine, unconverted mono alcohol and unconverted acid catalyst, into the preceding esterification device (9, 171, 173, 175, 179).

60. The device according to claim 59, wherein the feeding (181, 183, 193, 195) passes through a drying device (197) in order to remove at least one of water and mono alcohol from the heavy phase.

61. The device according to claim 60, wherein the drying device (197) serves as a distillation device for the evaporation of a water-mono alcohol mixture, or as a molecular sieve or micro filter to remove water as a permeate.

62. The device according to claim 59, wherein the feeding (181, 183, 193, 195) is provided with a subsidiary pipe (203, 205) for conveying a partial flow of the heavy phase into the same esterification device (171, 173, 175, 179, 11).

63. The device according to claim 50, wherein the esterification devices (9, 171, 173, 175, 179, 11) are connected with the transesterification unit (5) via at least one feeding (47) for conveying the products formed in the esterification devices (9, 11) into the transesterification device (5).

64. The device according to claim 50, wherein the transesterification unit (5) is equipped with 2 to 4 transesterification devices (15, 17) arranged consecutively and connected with each other.

65. The device according to claim 64, wherein the transesterification devices (15, 17) are designed as columns.

66. The device according to claim 64, wherein the transesterification devices (15, 17) are connected with the purification unit (6) via at least one feeding (105, 106) for conveying the products formed in the transesterification devices (15, 17) into the purification unit (6).

67. The device according to claim 50, wherein the purification unit (6) is equipped with at least 4 separators (71, 73, 75, 77), arranged consecutively to and connected with each other, and a drying device (159) for the purification of the obtained product.

68. The device according to claim 67, wherein the separators (71, 73, 75, 77) are connected with the drying device (159) via a feeding (57) for conducting the products that were separated from the base catalyst, mono alcohol, acid catalyst and dragging agent in the separators (71, 73, 75, 77) into the drying device (159).

69. The device according to claim 67, wherein the drying device (159) is equipped with an outlet conduit (163) for the end products purified in the drying device (159).

70. The device according to claim 67, wherein the separators (71, 73, 75, 77) are connected with the purification unit (8) via the feedings (107, 109) for conducting the mixture of base catalyst, mono alcohol, acid catalyst and dragging agent separated in the separators (71, 73 75, 77) into the purification unit (8).

71. The apparatus as claimed in claim 50, wherein the purification unit (8) comprises at least one drying apparatus (97), one acidification apparatus (103), one separator (113), one rectification apparatus (117), one distillation apparatus (131), one thin-film evaporation apparatus (139) and one filtration apparatus (147).

72. The device according to claim 50, wherein the esterification devices (9, 11) are connected with the drying device (97) via at least one feeding (95) for conducting a mixture comprising unconverted C₁- to C₄-mono alcohol, acid catalyst and dragging agent, obtained in the esterification devices via phase separation, into the drying device (97).

73. The device according to claim 72, wherein the drying device (97) is connected with the rectification device (117) via afeeding (125) for conveying the C₁- to C₄-mono alcohol separated in the drying device into the rectification device (117).

74. The device according to claim 72, wherein the drying device (97) is connected with the acidification device (103) via a feeding (101) for conducting the mixture of acid catalyst, conveying agent and traces of C₁- to C₄-mono alcohol, separated in the drying device (97), into the acidification device (103).

75. The device according to claim 71, wherein the acidification device (103) is connected with the separators (71,73,75, 77) via the feedings (107, 109).

76. The device according to claim 74, wherein the acidification device (103) is connected with the separator (113) via a feeding (111) for conveying the mixture that was acidified in the acidification device (103) into the separator (113).

77. The device according to claim 76, wherein the separator (113) is connected with the esterification unit (3) via a feeding (155) for re-conducting the fatty acid separated in the separator (113) into the esterification unit (3).

78. The device according to claim 76, wherein the separator (113) is connected with the rectification device (117) via a feeding (115) for conveying the water-containing mixture of acid catalyst, base catalyst and traces of the mono alcohol formed in the separator (113) into the rectification device (117).

79. The device according to claim 78, wherein the rectification device (117) is connected with the esterification unit (3) via a feeding (127) for re-conducting the mono alcohol purified in the rectification device (117) into the esterification unit (3).

80. The device according to claim 78, wherein the rectification device (117) is connected via a feeding (119) with the evaporation device (121).

81. The device according to claim 80, wherein the evaporation device (121) is equipped with a feeding (123) serving to convey the water purified in the evaporation device (121).

82. The device according to claim 80 or 81, wherein the evaporation device (121) is connected with the drying device (131) and the distillation device (135) via feedings (129, 133) for conveying the mixture of glycerin, acid catalyst and base catalyst, separated in the evaporation device (121), into the device (135).

83. The device according to claim 82, wherein the distillation device (135) is connected with the thin-layer-evaporation device (139) via afeeding (137) for conveying the acid and base catalysts separated in the device (135) into the evaporation device (139).

84. The device according to claim 83, wherein the thin-layer-evaporation device (139) is equipped with a pipe (141) serving as adischarge for the conversion product of the base and acid catalyst that is suitable for use as a fertilizer.

85. The device according to claim 83, wherein the distillation device (135) is connected with the filtration device (147) via a feeding (145) for conveying the glycerin into the filtration device (147).

86. The device according to claim 85, wherein the filtration device (147) is connected with the esterification unit (3) via a feeding (149) in order to re-cycle a partial flow of the glycerine purified in the filtration device so that it can be reused as dragging agent.

87. The method according to claim 1, wherein the alkyl esters of higher fatty acids comprise biodiesel.

88. The method according to claim 36, wherein the pH-conditioned water or buffered water is condensation water or de-ionized water.

89. The method according to claim 44, wherein the water content of the purified mono alcohol is 0.1%.
